(19) United States
(12) Patent Application Publication
(10) Pub. No.: US 2019/0343634 A1
(43) Pub. Date: Nov. 14, 2019

(54) RADIAL ARM TISSUE ANCHOR FOR MINIMALLY INVASIVE HEART VALVE REPAIR

(71) Applicant: NeoChord, Inc., St. Louis Park, MN (US)

(72) Inventors: Graham Garvin, Redwood City, CA (US); Tim Crowley, Arvada, CO (US); Joel Helgerson, Boulder, CO (US); Tom Broome, Mound, MN (US); Daryl Edmiston, Draper, UT (US)

(21) Appl. No.: 16/406,799

(22) Filed: May 8, 2019

Related U.S. Application Data
(60) Provisional application No. 62/669,123, filed on May 9, 2018.

(51) Int. Cl. A61F 2/24; A61B 17/04

(52) U.S. Cl. CPC A61F 2/2457; A61F 2/2466; A61B 17/0469; A61F 2220/0016; A61F 2230/0093; A61B 2017/0412; A61B 2017/042; A61B 2017/0409; A61B 2017/0464

(57) ABSTRACT

Disclosed herein are various embodiments of anchors configured to be inserted into a heart wall of a patient to anchor a suture as an artificial chordae under an appropriate tension for proper valve function. Each of the disclosed anchor embodiments transitions from a first position for delivery of the anchor to the heart wall to a second position for insertion of the anchor into the heart wall. In some embodiments, it is the transition to the second position that provides the necessary insertion force for inserting the anchor into the heart muscle sufficient to retain the anchor from accidental withdrawal from the heart wall during normal valve operation (e.g., when a valve leaflet pulls on the suture attached to the anchor during systole). Such anchors are particularly suitable for use in intravascular, transcatheter procedures as described above given the inherent difficulties in providing sufficient force for insertion of an anchor into the heart wall with a flexible catheter.

### RELATED APPLICATION

This application claims the benefit of U.S. Provisional Application No. 62/669,123 filed May 9, 2018, which is hereby fully incorporated herein by reference.

### TECHNICAL FIELD

The present invention relates to minimally invasive delivery of a suture. More particularly, the present invention relates to anchoring of a suture as an artificial chordae tendineae for a flailing or prolapsing leaflet in a beating heart.

### BACKGROUND

The mitral and tricuspid valves inside the human heart include an orifice (annulus), two (for the mitral) or three (for the tricuspid) leaflets and a subvalvular apparatus. The subvalvular apparatus includes multiple chordae tendineae, which connect the mobile valve leaflets to muscular structures (papillary muscles) inside the ventricles. Rupture or elongation of the chordae tendineae results in partial or generalized leaflet prolapse, which causes mitral (or tricuspid) valve regurgitation. A commonly used technique to surgically correct mitral valve regurgitation is the implantation of artificial chordae (usually 4-0 or 5-0 Gore-Tex sutures) between the prolapsing segment of the valve and the papillary muscle.

This was traditionally an open heart operation generally carried out through a median sternotomy and requiring cardiopulmonary bypass with aortic cross-clamp and cardioplegic arrest of the heart. Using such open heart techniques, the large opening provided by a median sternotomy or right thoracotomy enables the surgeon to see the mitral valve directly through the left atriotomy, and to position his or her hands within the thoracic cavity in close proximity to the exterior of the heart for manipulation of surgical instruments, removal of excised tissue, and/or introduction of an artificial chordae through the atriotomy for attachment within the heart. However, these invasive open heart procedures produce a high degree of trauma, a significant risk of complications, an extended hospital stay, and a painful recovery period for the patient. Moreover, while heart valve surgery produces beneficial results for many patients, numerous others who might benefit from such surgery are unable or unwilling to undergo the trauma and risks of such techniques.

Techniques for minimally invasive thoracoscopic repair of heart valves while the heart is still beating have also been developed. U.S. Pat. No. 8,465,500 to Speziali, which is incorporated by reference herein, discloses a thoracoscopic heart valve repair method and apparatus. Instead of requiring open heart surgery on a stopped heart, the thorascopic heart valve repair methods and apparatus taught by Speziali utilize fiber optic technology in conjunction with transesophageal echocardiography (TEE) as a visualization technique during a minimally invasive surgical procedure that can be utilized on a beating heart. More recent versions of these techniques are disclosed in U.S. Pat. Nos. 8,758,393 and 9,192,374 to Zentgraf, which disclose an integrated device that can enter the heart chamber, navigate to the leaflet, capture the leaflet, confirm proper capture, and deliver a suture as part of a mitral valve regurgitation (MR) repair. These minimally invasive repairs are generally performed through a small, between the ribs access point followed by a puncture into the ventricle through the apex of the heart. Although far less invasive and risky for the patient than an open heart procedure, these procedures still require significant recovery time and pain.

Some systems have therefore been proposed that utilize a catheter routed through the patient's vasculature to enter the heart and attach a suture to a heart valve leaflet as an artificial chordae. While being less invasive than the approaches discussed above, transcatheter heart valve repair can provide additional challenges. For example, with all artificial chordae replacement procedures, in addition to inserting a suture through a leaflet, the suture must also be anchored at a second location, such as at a papillary muscle in the heart, with a suture length, tension and positioning that enables the valve to function naturally. If the suture is too short and/or has too much tension, the valve leaflets may not properly close. Conversely, if the suture is too long and/or does not have enough tension, the valve leaflets may still be subject to prolapse. Proper and secure anchoring of the suture away from the leaflet is therefore a critical aspect of any heart valve repair procedure for inserting an artificial chordae. In the case of transcatheter procedures, such anchoring can be difficult because it can be difficult for the flexible catheter required for routing through the patient's vasculature to apply sufficient force to stably insert traditional suture anchors into, e.g., the myocardium.

### SUMMARY

Disclosed herein are various embodiments of anchors configured to be inserted into a heart wall of a patient to anchor a suture as an artificial chordae under an appropriate tension for proper valve function. Each of the disclosed anchor embodiments transitions from a first position for delivery of the anchor to the heart wall to a second position for insertion of the anchor into the heart wall. In some embodiments, it is the transition to the second position that provides the necessary insertion force for inserting the anchor into the heart muscle sufficient to retain the anchor from accidental withdrawal from the heart wall during normal valve operation (e.g., when a valve leaflet pulls on the suture attached to the anchor during systole). Such anchors are particularly suitable for use in intravascular, transcatheter procedures as described above given the inherent difficulties in providing sufficient force for insertion of an anchor into the heart wall with a flexible catheter.

In one embodiment, a method of anchoring a suture in a patient's heart as an artificial chordae includes intravascularly accessing a patient's heart and inserting a suture into a heart valve leaflet of the patient's heart. A portion of the suture can be attached to a radial arm tissue anchor or “spider anchor” including an anchor body and one or more anchor tines. The spider anchor can be inserted into the patient's heart intravascularly with an anchor delivery catheter with the spider anchor in a delivery position having the anchor tine(s) extending generally axially with respect to the anchor body such that the spider anchor fits within the anchor delivery catheter. The spider anchor can then be advanced out of the anchor delivery catheter and into the heart wall such that the spider anchor transitions from the delivery position into an anchoring position with the anchor tines flaring or curving outwardly relative to the anchor body as the spider anchor is advanced into the heart wall into the anchoring position. In some embodiments, the transition from the delivery position to the anchoring position provides a force sufficient to cause the anchor tines to penetrate into the heart wall. The anchor delivery catheter can then be removed from the heart leaving the spider anchor in the heart with the suture extending between the leaflet and the spider anchor as an artificial chordae.

In one embodiment, an anchor is configured to be implanted into a patient's heart wall to anchor a suture extending from a valve leaflet of the heart as an artificial chordae. The anchor can include an anchor shaft and one or more anchor tines extending from a distal end of the anchor shaft. Anchor tines can be configured for delivery to the heart wall in a delivery configuration generally axially aligned with the anchor shaft such that the anchor shaft and anchor tines can be contained within an anchor delivery catheter. Anchor tines can further be configured to flare or curve outwardly from the delivery configuration into an anchoring configuration when advanced out of the anchor delivery catheter and into the heart wall to retain the anchor within the heart wall. In one embodiment, anchor tines automatically transition to the anchoring configuration upon exiting the anchor delivery catheter due to the anchor tines being pretreated to have a shape memory in the anchoring configuration.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A-1K depict various steps in a method of anchoring a suture in a beating heart of a patient to function as an artificial chordae according to an embodiment.

FIG. 2 depicts a radial arm tissue anchor for an artificial chordae according to an embodiment.

FIG. 3 depicts a radial arm tissue anchor for an artificial chordae according to an embodiment.

FIG. 4 depicts a radial arm tissue anchor for an artificial chordae according to an embodiment.

FIG. 5 depicts a radial arm tissue anchor for an artificial chordae according to an embodiment.

FIGS. 6A-6B depict a radial arm tissue anchor for an artificial chordae according to an embodiment.

FIGS. 7A-7D depict insertion of a radial arm tissue anchor for an artificial chordae into a body of a patient according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure is generally directed to anchoring of sutures inserted as artificial chordae into one or more heart valve leaflets through an intravascular, transcatheter approach. A heart valve leaflet may be captured and a suture inserted through the leaflet in any manner known in the art. One such leaflet capture catheter and procedure is disclosed in copending U.S. Utility patent application Ser. No. 16/363,701, which is hereby incorporated by reference herein. Another transcatheter procedure for inserting an artificial chordae is disclosed in U.S. Patent Publication No. 2016/0143737, which is hereby incorporated by reference herein.

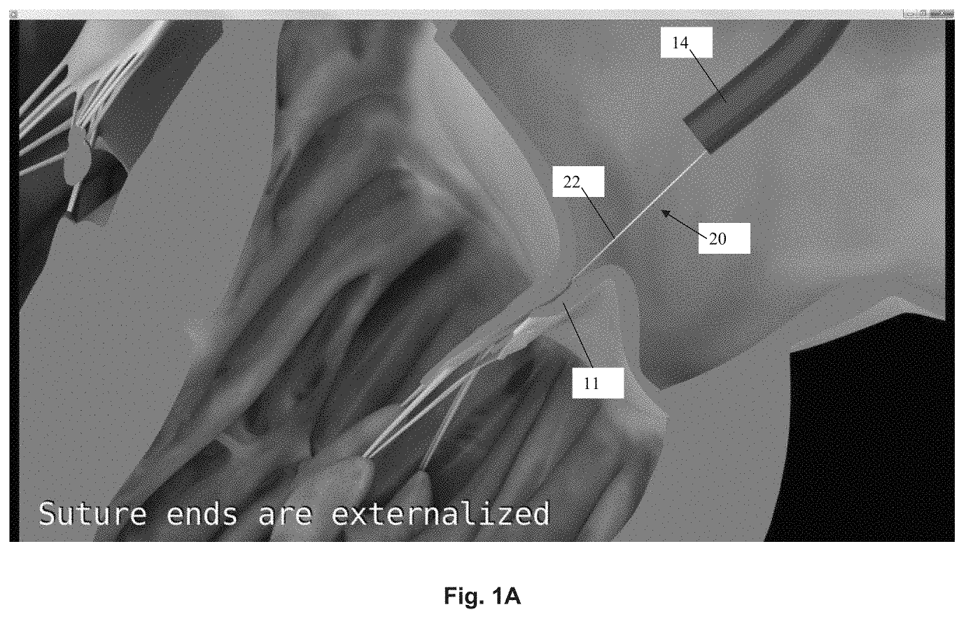

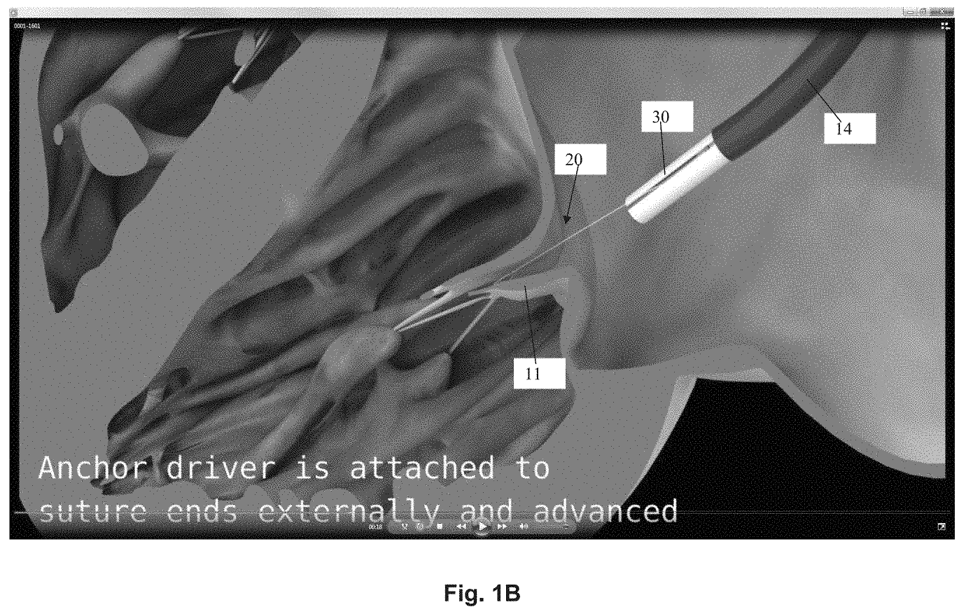

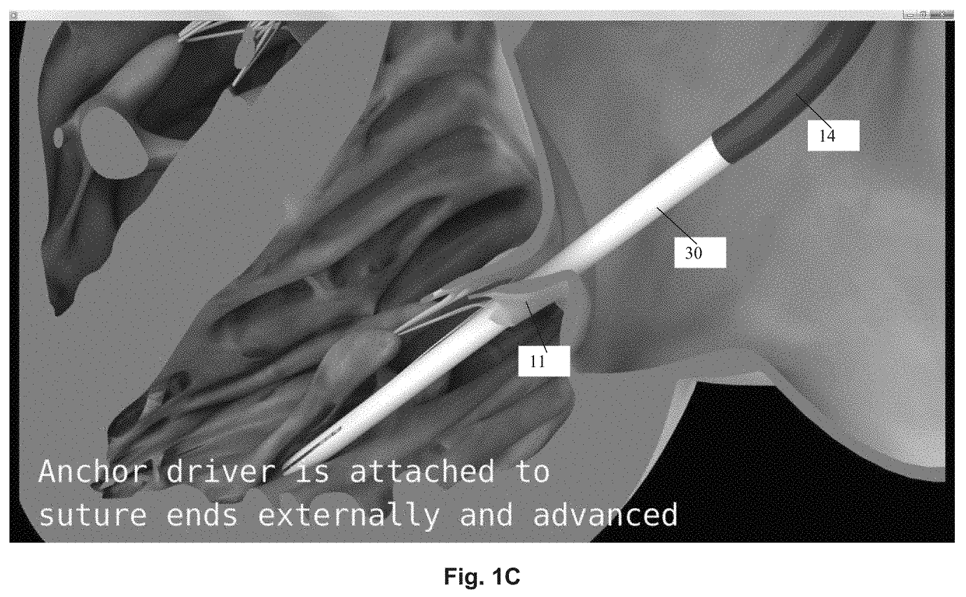

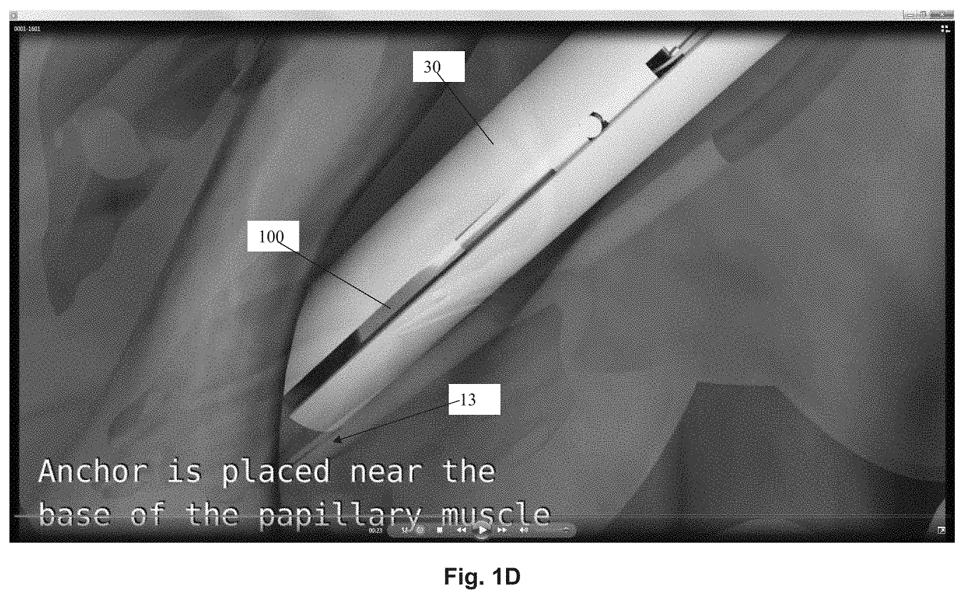

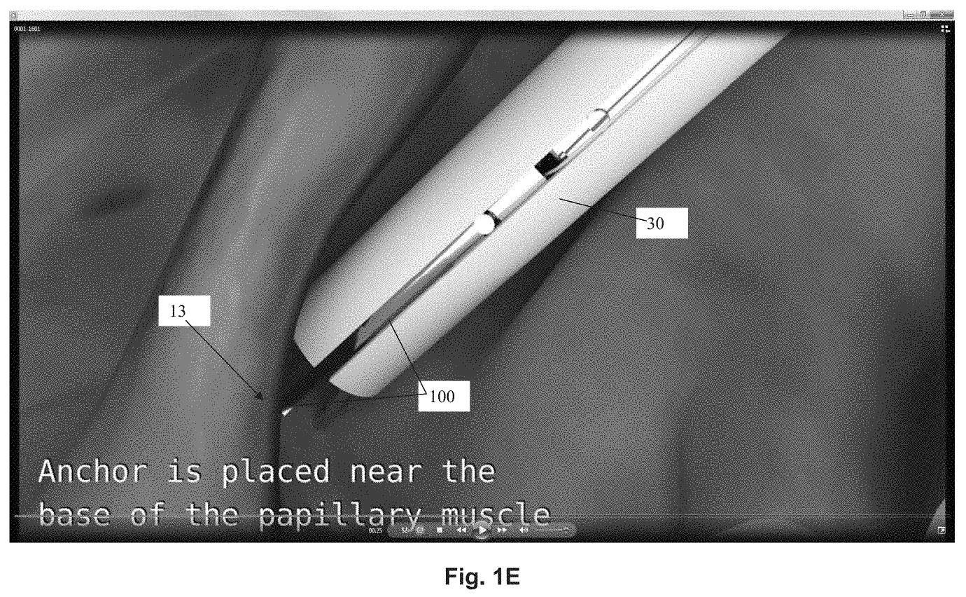

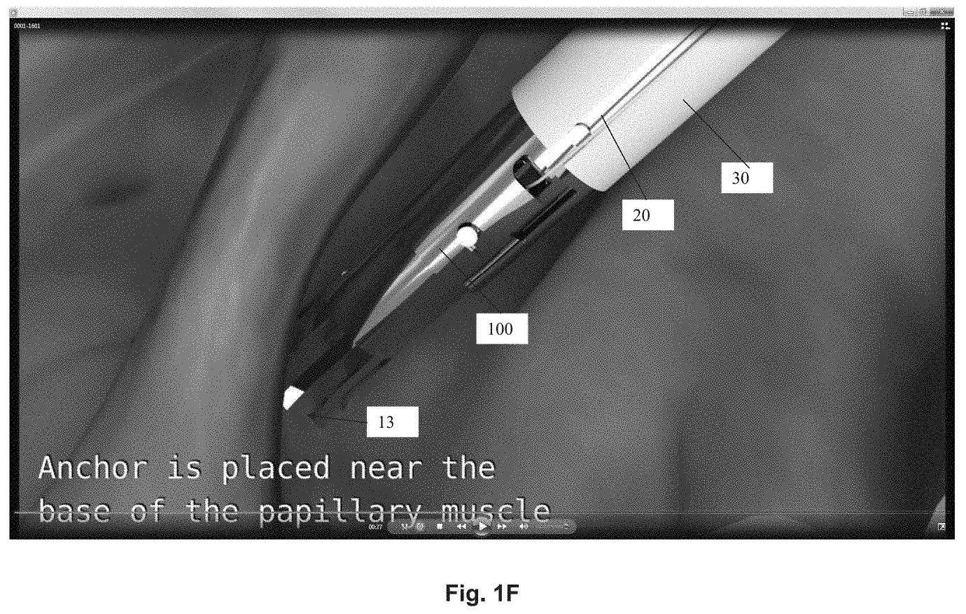

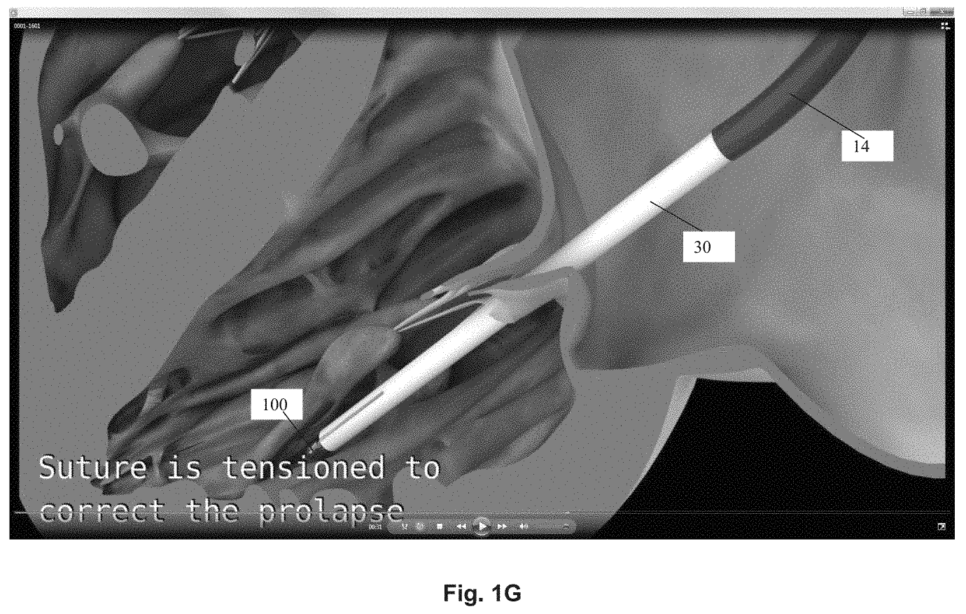

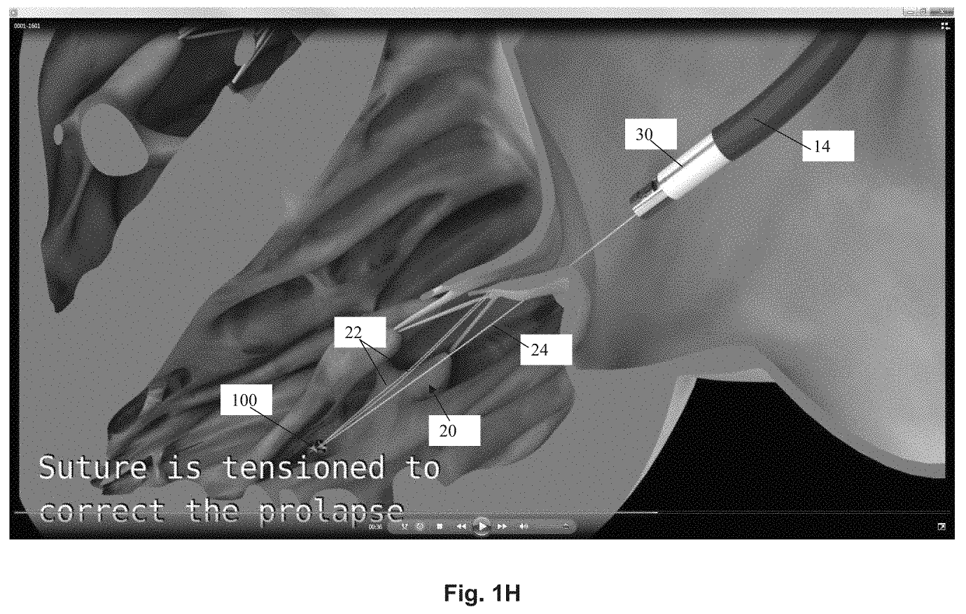

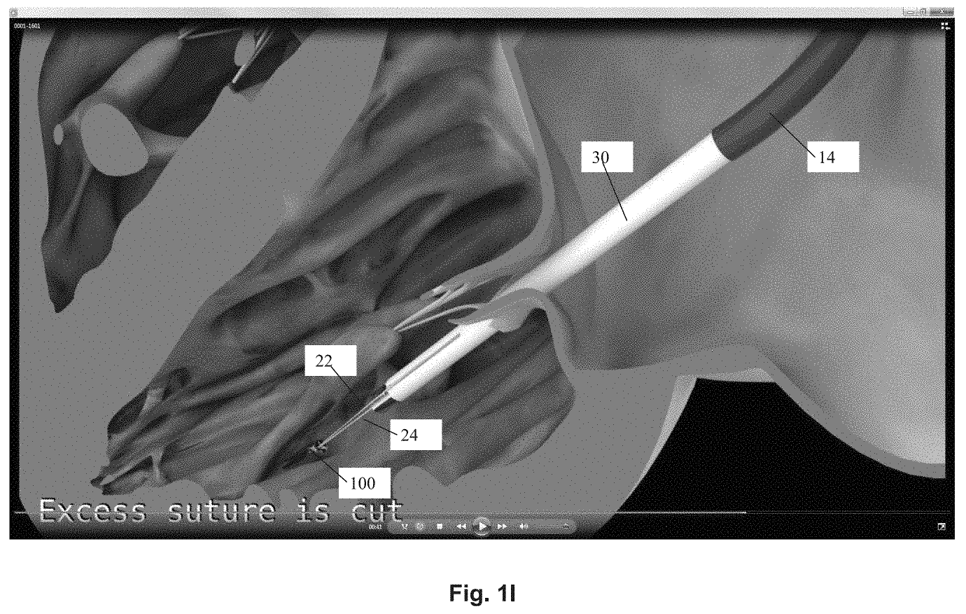

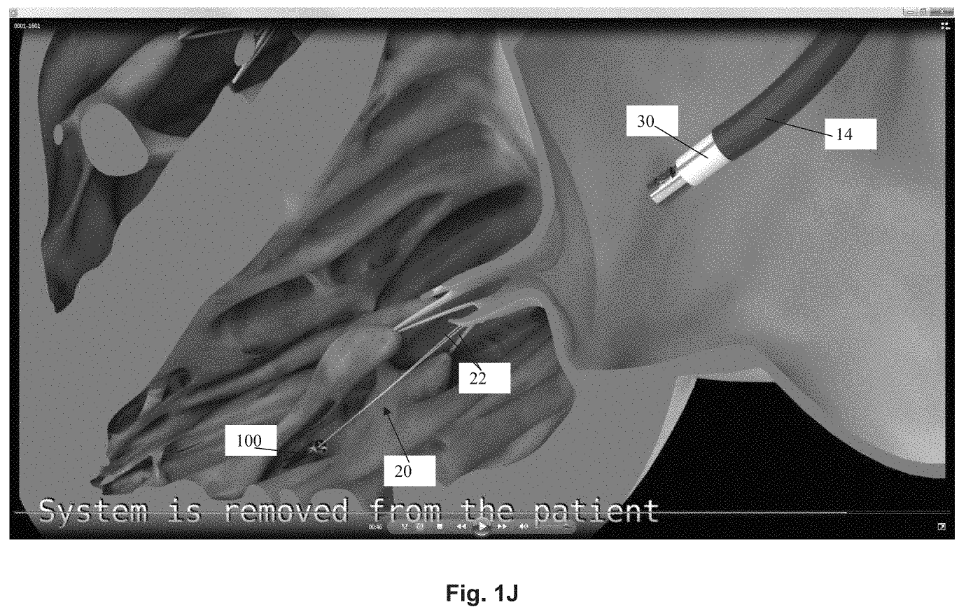

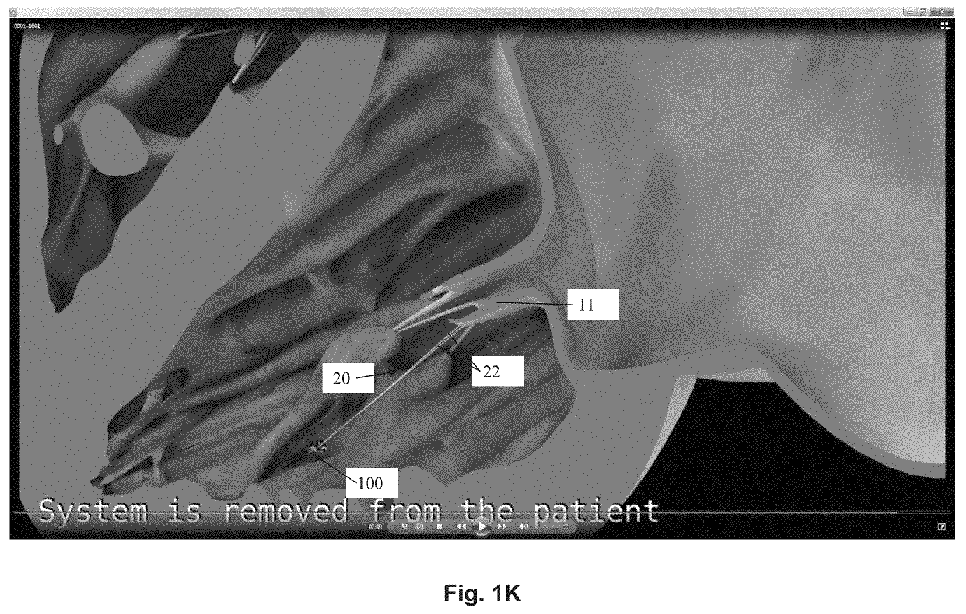

Referring to FIGS. 1A-1K, a procedure for anchoring a suture inserted as an artificial chordae in a transcatheter procedure on a beating heart of a patient following insertion of the suture into a leaflet is schematically depicted. In this embodiment, a loop of suture has been inserted through the leaflet and the two free ends of the suture then inserted through the loop to form a girth hitch knot around the edge of the leaflet. Further detail regarding attaching a suture to a leaflet in this manner can be found in U.S. Patent Publication No. 2017/0290582, which is hereby incorporated by reference herein.

Following insertion of the suture 20 into the leaflet 11, the deployment catheter used to insert the suture is withdrawn through the guide catheter 14 and the two free ends 22 of the suture 20 are also withdrawn external to the body. The suture ends 22 are then attached to an anchor contained in an anchor driving catheter 30. Alternatively, the anchor could be pre-attached to the suture prior to insertion of the suture into the leaflet. The anchor driving catheter 30 is inserted into the guide catheter 14, routed through the catheter into the body and advanced passed the leaflet 11 to the heart wall 13 below the valve at, for example, a papillary muscle as shown in FIGS. 1B-1D. The anchor driving catheter 30 is then used to insert the anchor 100 into the myocardium as shown in FIGS. 1D-1G and as described in more detail below.

After insertion of the anchor 100 into the heart tissue, the anchor driving catheter 30 is withdrawn to a position superior of the valve as shown in FIG. 1H and the length and tension of the suture ends 22 extending from the leaflet 11 are tested and adjusted until it is determined that normal valve function has been achieved. This determination can be made through use of ultrasonic imaging, for example. The tension is adjusted through a tensioning strand 24 of the suture depicted in FIG. 1H. Once the proper length and tension has been determined using, for example, transesophageal echocardiography or other non-invasive methods, the anchor driving catheter 30 is advanced back down along the tensioning strand 24 and to sever the strand at the anchor 100. The entire catheter system, including the anchor driving catheter 30 and the guide catheter 14 is then withdrawn from the patient's body. Referring to FIG. 1K, the suture 20 remains in the body extending between the leaflet 11 and the anchor 100 to function as an artificial chordae tendineae.

Disclosed herein are various embodiments of an anchor that can be employed in procedures such those described above to anchor a suture as an artificial chordae. Such anchors maintain positioning and length of the suture (i.e., tension) to ensure proper leaflet functionality during the cardiac cycle.

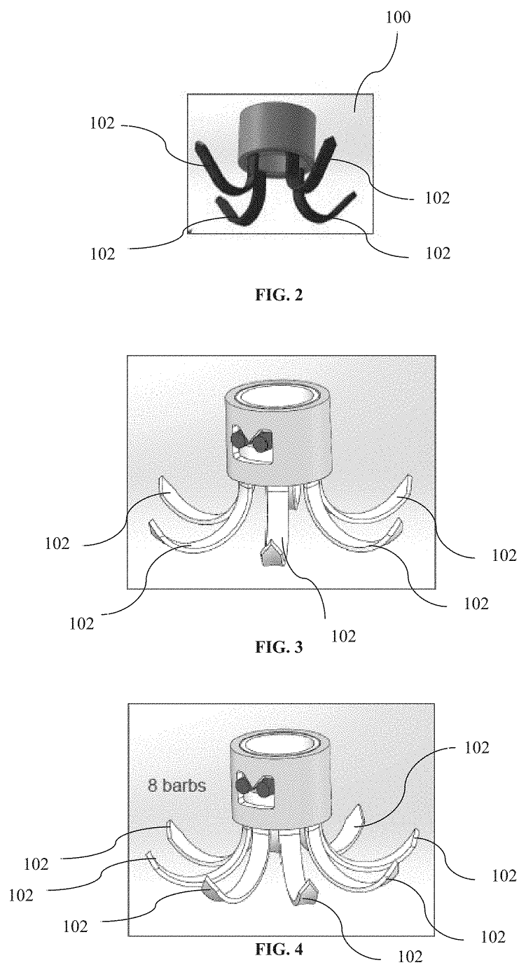

Referring now to FIG. 2-4, one embodiment of a radial arm tissue anchor 100 or “spider anchor” for anchoring a suture as an artificial chordae is depicted. Spider anchor 100 generally includes a plurality of elongate anchor tines or radial arms 102 extending outwardly away from an anchor body or shaft 104. In one embodiment, spider anchor 100 can further include one or more suture bars 106 that aid in adjusting a tension and/or length of one or more sutures connected to spider anchor. Examples of such tensioning mechanisms can be found in U.S. Provisional Patent Application No. 62/669,115, entitled Suture Length Adjustment for Minimally Invasive Heart Valve Repair, which is hereby incorporated by reference herein in its entirety.

According to embodiments, spider anchor 100 can include various numbers of tines 102. For example, FIG. 2 depicts spider anchor 100 having four tines. FIG. 3 depicts spider anchor 100 having six tines and FIG. 4 depicts spider anchor 100 having eight tines. According to various embodiments, spider anchors as disclosed herein can include as few as three tines and as many as twelve or more tines.

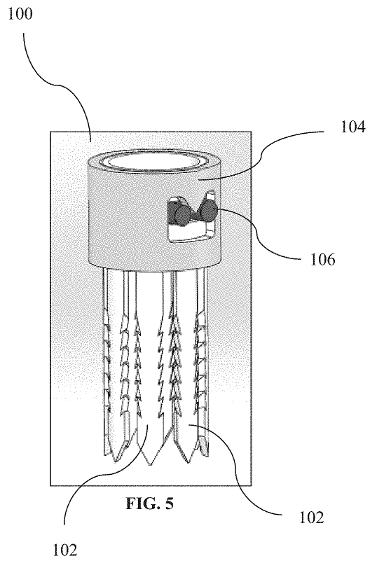

Tines 102 can each include an elongate body 108 and a distal tip 110. Distal tip 110 is sharpened to aid in insertion of tines 102 into heart tissue. According to some embodiments, as depicted in FIGS. 2-4, elongate body 108 of anchor tines 102 may be generally smooth. According to alternative embodiments, as illustrated in FIG. 5, elongate body 108 of anchor tines 102 may be serrated. Such a serrated configuration further secures the anchor 100 into the body by increasing the force that would be required to pull the tines 102 out of the heart muscle. According to some embodiments, spider anchor 100 may be made of a shape memory material such as, for example, Nitinol. In such embodiments tines 102 can be formed with anchor body 104 in a monolithic construction. In some embodiments, tines 102 and body 104 can be formed by laser cutting tines 102 from a Nitinol tube. In other embodiments, tines 102 can be formed from a laser cut tube and a separate body 104 can surround and/or be attached to the remaining tube portion from which the tines extend.

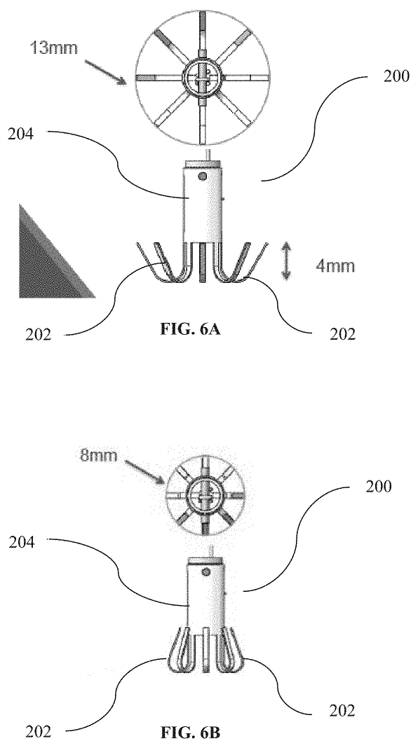

FIGS. 6A-6B depict a radial arm tissue anchor or spider anchor 200 according to another embodiment. Anchor 200 similarly includes an anchor body or shaft 204 with a plurality of anchor tines 202 extending therefrom. In this embodiment, anchor tines 202 are unitarily formed with anchor body 204 in a single monolithic construction, as discussed above. FIGS. 6A-6B further depict one embodiment of a heat treatment process for providing shape memory to anchor tines 202 that includes a two stage process. FIG. 6A depicts the first stage of the process in which the tines 202 are partially curved with respect to anchor body 204. In one embodiment of this configuration, the outer diameter of the anchor, as determined by how for outwardly the anchor tines 202, is 13 millimeters. FIG. 6B depicts a second stage of the process after the tines 202 have been heat set in the first stage that further curves the tines 202 with respect to body 204. In one embodiment of this configuration, the outer diameter of the anchor is 8 millimeters. In such embodiments, tines are heat set to a curved geometry such that upon deployment from a delivery catheter, as discussed below, tines 202 lose restraint from the delivery catheter and regain their curved shape, securing them in the muscle.

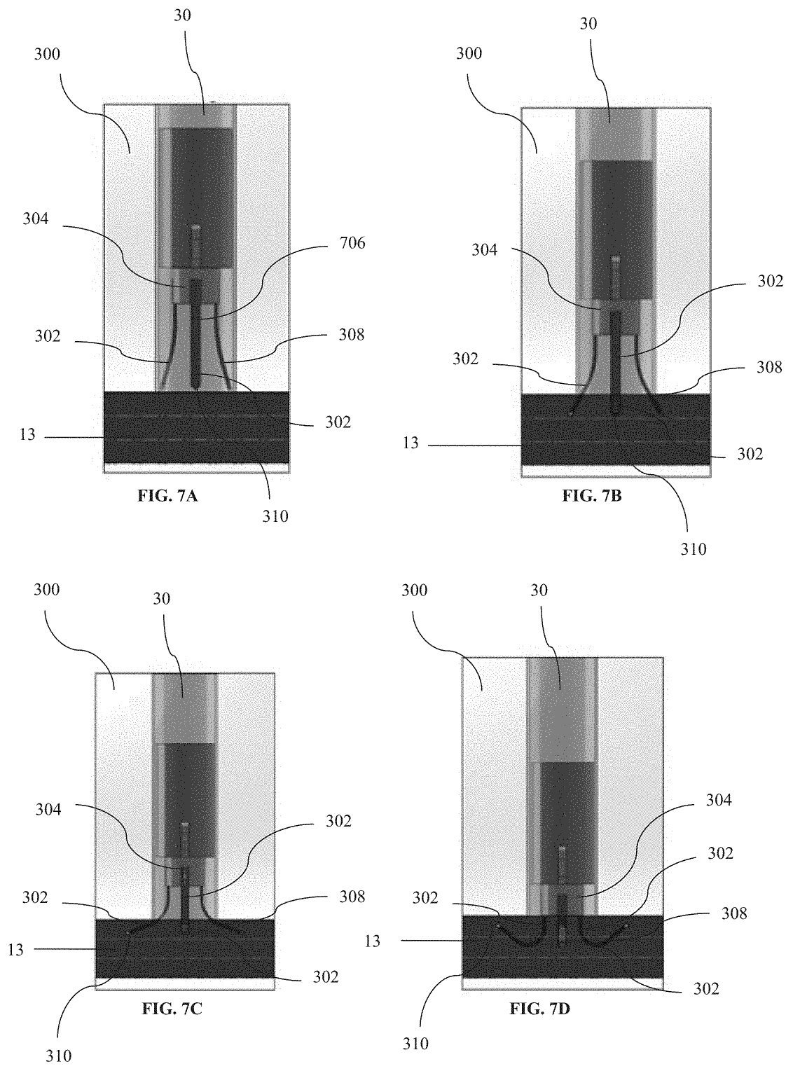

FIGS. 7A-7D depict a radial arm tissue anchor or spider anchor 300 according to a further embodiment. Spider anchor 300 includes an anchor body or shaft 304 having one or more anchor tines 302 extending distally therefrom. A suture (not pictured) can be attached to spider anchor 300 within anchor body 304. Anchor tines 302 can be unitarily formed with anchor body 304 in a monolithic construction as described herein. Each tine 302 can include an elongate arm 308 that unitarily extends from a distal end of the anchor body 304 of spider anchor 300. Tines 302 each further include a pointed tip 310 at a distal end of each elongate arm 308. As discussed above, tines 302 can each be biased outwardly towards an anchoring configuration.

Still referring to FIGS. 7A-7D, these figures further depict a procedure for inserting one embodiment of a radial arm tissue anchor or spider anchor 300 into a heart wall. FIG. 7A depicts a delivery position or configuration of the spider anchor 300 for when the anchor 300 and anchor driving catheter 30 are guided through the guide catheter 14 (not pictured) and the anchor driving catheter 30 is advanced adjacent the myocardium. In this configuration, the anchor tines 302 are generally axially or longitudinally aligned with the anchor shaft 304, which enables the anchor 300 to be contained within the anchor driving catheter 30 so that the anchor 300 can be routed through the guide catheter 14 (not pictured).

Adjacent the myocardium 13, the anchor tines 302 are advanced out of the anchor driving catheter 30 and as the anchor tips 310 are driven into the myocardium, the elongate arms 308 of the anchor tines 302 flare or curve outwardly with respect to the anchor shaft 304 as shown in FIG. 7B. The anchor tines 302 automatically flare when advanced out of the anchor driving catheter 30 when no longer constrained in the delivery position by the anchor driver 30 due to their heat set, shape memory curved form, which provides the necessary force to insert the anchor tines 302 into the heart wall 13. As the anchor tines 302 are further advanced out of the guide catheter 30 and driven into the heart muscle, the anchor tines 302 can continue to curve or flare outwardly and upwardly into a final, anchoring position that inhibits inadvertent removal of the anchor tines 302. The anchor 300 then remains in the body with one or more sutures extending between the anchor 300 and a leaflet as an artificial chordae. In one embodiment, anchor shaft 304 includes a tensioning mechanism through which a tension on the suture can be adjusted, as previously referenced herein.

Disclosed herein are various embodiments of anchors configured to be inserted into a heart wall of a patient to anchor a suture as an artificial chordae under an appropriate tension for proper valve function. Each of the disclosed anchor embodiments transitions from a first position for delivery of the anchor to the heart wall to a second position for insertion of the anchor into the heart wall. In some embodiments, it is this transition that provides the necessary insertion force for inserting the anchor into the heart muscle sufficient to retain the anchor from accidental withdrawal from the heart wall during normal valve operation (e.g., when a valve leaflet pulls on the suture attached to the anchor during systole). Such anchors are particularly suitable for use in intravascular, transcatheter procedures as described above given the inherent difficulties in providing sufficient force for insertion of an anchor into the heart wall with a flexible catheter.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

1. A method of anchoring a suture in a heart of a patient as an artificial chordae, comprising:
intravascularly accessing the heart;
inserting a suture into a heart valve leaflet of the heart;
attaching a portion of the suture to an anchor, the anchor including an anchor body and a plurality of anchor tines extending distally from the anchor body, each anchor tine including an elongate body and a distal tip;
advancing the anchor into the heart with an anchor delivery catheter with the anchor in a delivery position having the anchor tines extending generally axially with respect to the anchor body such that the anchor fits within the anchor delivery catheter;
positioning the anchor adjacent a heart wall of the heart;
advancing the anchor out of the anchor delivery catheter and into the heart wall such that the anchor transitions from the delivery position into an anchoring position as the anchor is advanced into the heart wall, the anchor tines curving generally outwardly with respect to the anchor body in the anchoring position, and wherein the transition from the delivery position to the anchoring position provides a force sufficient to cause the anchor tines to penetrate into the heart wall; and
removing the anchor delivery catheter from the heart leaving the anchor in the heart with the suture extending between the leaflet and the anchor as an artificial chordae.

2. The method of claim 1, wherein the anchor transitions from the delivery position into the anchoring position automatically when the anchor tines are advanced out of the anchor delivery catheter.

3. The method of claim 2, wherein the anchor comprises a shape memory material that automatically transitions into the anchoring position when not constrained by the anchor delivery catheter.

4. The method of claim 3, wherein the shape memory material is heat set into the anchoring position.

5. The method of claim 1, wherein the anchor transitions from the delivery position into the anchoring position by the anchor tines curving with respect to the anchor shaft.

6. The method of claim 5, wherein the anchor tines flare outwardly with respect to the anchor shaft.

7. The method of claim 1, wherein the anchor tines are unitarily formed with the anchor shaft as a single monolithic construction.

8. The method of claim 1, wherein the plurality of anchor tines are radially arranged around a distal end of the anchor shaft.

9. The method of claim 1, further comprising adjusting a tension of the suture.

10. The method of claim 1, wherein the elongate body of one or more of the plurality of anchor tines is serrated.

11. An anchor configured to be implanted into a heart wall of a heart of a patient to anchor a suture extending from a valve leaflet of the heart as an artificial chordae, the anchor comprising:
an anchor shaft; and
a plurality of anchor tines extending from a distal end of the anchor shaft, each anchor tine including an elongate body and a distal tip, wherein the anchor tines are configured for delivery to the heart wall in a delivery configuration generally axially aligned with the anchor shaft such that the anchor shaft and anchor tines can be contained within an anchor delivery catheter, and wherein the anchor tines are configured to transition from the delivery configuration into an anchor configuration when advanced out of the anchor delivery catheter and into the heart wall, the anchor tines being curved generally outwardly with respect to the anchor shaft in the anchor configuration to retain the anchor within the heart wall.

12. The anchor of claim 11, wherein the anchor tines are configured to transition from the delivery configuration into the anchoring configuration automatically when the anchor tines are advanced out of the anchor delivery catheter.

13. The anchor of claim 12, wherein the anchor tines comprise a shape memory material that automatically transitions into the anchoring position when not constrained by the anchor delivery catheter.

14. The anchor of claim 13, wherein the shape memory material is heat set into the anchoring position.

15. The anchor of claim 11, wherein the anchor tines transition from the delivery configuration into the anchoring configuration by the anchor tines flaring outwardly with respect to the anchor shaft.

16. The anchor of claim 11, wherein the elongate body of one or more of the plurality of anchor tines is serrated.

17. The anchor of claim 11, wherein the anchor tines are unitarily formed with the anchor shaft as a single monolithic construction.

18. The anchor of claim 11, wherein the plurality of anchor tines are radially arranged around a distal end of the anchor shaft.
